(19) United States
(12) Patent Application Publication
(10) Pub. No.: US 2008/0206877 A1
(43) Pub. Date: Aug. 28, 2008

(54) Reactors for selective enhancement reactions and methods of using such reactors

(75) Inventors: James E. Hutchison, Eugene, OR (US); Scott F. Sweeney, Eugene, OR (US); John A. Berglund, Eugene, OR (US); Amy E. Mahady, Eugene, OR (US)

Correspondence Address: KLARQUIST SPARKMAN, LLP, 121 SW SALMON STREET, SUITE 1600, PORTLAND, OR, 97204, US

(73) Assignees: The State of Oregon Acting By And Through The State Board Of Higher Education On Behalf Of; The University of Oregon

(21) Appl. No.: 11/710,148

(22) Filed: Feb. 22, 2007

(51) Int. Cl. C12M 1/00

(52) U.S. Cl. 436/55; 435/286.1

(57) ABSTRACT

Micro-reactors for selective enhancement of ligands by exponential enrichment (ISOS) include a reactant chamber defined in a fluoropolymer. A target exposure aperture controls a surface area, crystal face or orientation and surface features of a target that is exposed to a random mixture of candidate molecules. The surface area of the target can be selected based on number or concentration of candidate species to enhance candidate competition. The target surface can be formed by deposition of a thin film of a target material on a rigid substrate such as a glass plate. Selected exposure areas are typically substantially smaller than a characteristic cross sectional area of a reactant chamber volume and can be at least as small as 0.1 mm².

### ACKNOWLEDGEMENT OF GOVERNMENT SUPPORT

This work was sponsored by an agency of the United States government under Air Force Research Agreement FA8650-05-1-5041, and the U.S. government has certain rights in the invention.

### FIELD

The disclosure pertains to methods and apparatus for selective enhancement reactions.

### BACKGROUND

Advancements in biotechnology as well as materials and nanoscience have permitted the development of sophisticated methods for selection of biomolecules that exhibit specific binding to various materials of technological relevance. Biomolecules selected for such specific binding can permit the fabrication of novel optical, sensor, electronic and magnetic materials and also permit the assembly of these materials for a number of emerging applications. For example, the specific binding of peptides to selected semiconductor surfaces has been proposed as a method for the assembly of nanocrystals. Some examples of devices and materials that can be fabricated and assembled using biological reagents and scaffolds are described in, for example, Eaton et al., U.S. Patent Application Pub. 2005/0136439, Hutchison et al., U.S. Patent Application Pub. 2006/0081835, Sau et al., J. Nanoparticle Res. 3:257-262 (2001), Sinensky and Belcher, PCT Publication WO 2006/045071, Peelle et al., PCT Publication WO 2006/0465071, and Bruesehoff et al., Combinatorial Chemistry and High Throughput Screening 5:327-335 (2002).

Identification of biomolecules relevant for materials applications is generally based upon selection of a preferred biomolecule, for example RNA, DNA or peptide, by a repetitive competition for binding sites on a target surface. A mixture is exposed to a target in a first step, and some portion of the mixture is preferentially bound to the target for subsequent removal and amplification. This procedure can be repeated, with the biomolecules selected in a previous step presented to a target in a subsequent step so that each step tends to enhance material selectivity.

While methods have been developed for the screening of biomolecules relevant to materials applications, a significant challenge remains in identifying biomolecules with a high degree of surface specificity, for example crystallographic orientation or defect sites, under carefully controlled conditions of target concentration and temperature. Accordingly, improved methods and apparatus are needed.

### SUMMARY

Disclosed herein are methods and apparatus that provide highly controlled conditions for the selection of a predetermined number of biomolecule candidates for a predetermined number of binding sites on a surface. In some examples, reactors for selective enhancement reactions comprise a reactant chamber that includes a reagent introduction aperture and a target exposure aperture. The reagent introduction aperture has an area at least 100 times greater than an area of the target exposure area. In typical examples, the reactant chamber is defined by the reagent introduction aperture, the target exposure aperture, and reactant chamber walls, wherein the reactant chamber walls are formed of a material that tends to exhibit low affinity for candidate molecules situated in the reaction chamber. The reactant chamber walls can be conveniently formed of a fluoropolymer such as polychlorotrifluorethyene (PCTFE). In some examples, the target aperture has an area of less than about 1 mm², less than about 0.01 mm², or less than about 0.001 mm². In additional examples, a target support is configured to support a target surface for coupling to the target aperture, and a temperature sensor is thermally coupled to the target surface and the target support, and is responsive to the temperature of the target surface. In additional examples, a heating element is coupled to the target surface and is responsive to the temperature sensor so as to establish a temperature of the target surface. In further examples, at least two target exposure apertures of substantially different areas are provided.

Methods of selective enhancement comprise selecting a volume of a candidate mixture and exposing a target surface to the candidate mixture through an aperture. Typically the aperture area is less than a characteristic surface area of the candidate mixture, or less than about ⅕, 1/10, 1/100, or 1/1000 of the characteristic area, wherein the characteristic surface area is an area defined as a square of a dimension corresponding to a length of a cube having a volume that is the same as the volume of a candidate mixture. In additional examples, the aperture area is less than 1/1000 of the characteristic surface area. In further examples, a concentration of candidate molecules in the candidate mixture is selected based on the aperture area. In other examples, a number of candidate molecules in the candidate molecule mixture is selected based on the aperture area. In alternative examples, an aperture area is selected based on a concentration of candidates in the candidate mixture.

In additional examples, an aperture area is selected based on a number of candidates in the candidate mixture, or a plurality of aperture areas is used. In some examples, the target surface is a single crystal surface, and in a particular example, the target surface is a (111) or other surface of gold. In further representative examples, candidates attached to the target surface are extracted and amplified. A supplemental candidate mixture based on the amplified, extracted candidates is prepared, and the target surface is exposed to the supplemental candidate mixture. Additional supplemental mixtures can be similarly processed, until a satisfactory selectivity is obtained. In some examples, between about four and six such steps produce superior results.

Reactors for selective enhancement of candidate molecules include a candidate mixture chamber coupled to a target exposure aperture and a mixture introduction aperture. The chamber is defined by a tapered bore in a section of a cylinder, wherein a target aperture radius is less than 1/10 of a radius of the cylindrical section. In some examples, the tapered bore is conical. A support structure includes a top portion, a bottom portion, and at least one connector, wherein the bottom portion is configured to receive the cylindrical section and the top portion is configured to contact a cover plate so as to substantially seal the chamber from the mixture introduction aperture. The connector is configured to secure the top portion and the bottom portion. In some examples, the cylindrical section is a fluoropolymer such as polychlorotrifluorethyene. In additional examples, the cover is transparent.

These and other examples are described in further detail below with reference to the accompanying drawings.

### BRIEF DESCRIPTION OF THE DRAWINGS

FIG. 1A is a sectional view of a representative reaction vessel for selective enhancement situated at a target surface.

FIG. 1B is a plan view of the reaction vessel and the target surface of FIG. 1B.

FIG. 2 is a sectional view of a representative reaction vessel.

FIGS. 3-4 are plan views of representative reaction vessels.

FIG. 5 is a sectional view of a reaction vessel that includes an insert.

FIG. 6 is a plan view of a reaction vessel that includes a plurality of reaction apertures.

FIG. 7 is a plan view of a reaction vessel that includes a plurality of reaction volumes coupled to apertures of a fixed area.

FIG. 8 is a sectional view of a reactor that includes a removable insert that includes an aperture that defines a target exposure area.

FIG. 9 is a block diagram of a representative surface-area-controlled selection method.

### DETAILED DESCRIPTION

The methods and apparatus described herein can be applied to selection of oligonucleotide strands, typically short strands of DNA and/or RNA. Such strands selected from a random pool based on specific binding to a target species or surface are referred to herein as aptamers. Aptamers that are selected for preferred binding to gold surfaces are particularly useful in the formation of gold nanocrystals for a variety of applications. Aptamer binding to a specific crystal plane of gold tends to slow or halt crystal growth along that plane, so that a particular shape can be realized. Production of shaped nano-crystals is accomplished by, for example, immersing gold seed crystals in a solution containing the appropriate aptamer.

As used in this application and in the claims, the singular forms “a,” “an,” and “the” include the plural forms unless the context clearly dictates otherwise. Additionally, the term “includes” means “comprises.” Further, the term “coupled” means electrically, electromagnetically, mechanically, fluidically, or chemically coupled or linked based, and does not exclude the presence of intermediate elements between the coupled items.

The described systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art. In the examples described in detail below, In Vitro Selection on Surfaces (ISOS) is applied to the selection, replication, and amplification of RNA. The disclosed methods and apparatus can be applied to any biomolecule that can be replicated and amplified, and RNA is used only as an illustrative.

### In Vitro Selection on Surfaces

As generally described below, a selected area of a target surface is exposed to mixture of RNA fragments or other candidate molecules. Candidates that bind to the target surface are amplified and, in some cases, used to form a new mixture that is used for a subsequent target exposure. In some examples, a volume of a mixture of candidate molecules is exposed to a target surface through a target exposure aperture that limits the target surface area with which the candidate molecules can interact. A characteristic surface area of a mixture volume is a square of a length of a cube having the mixture volume. Typically, target exposure apertures have areas substantially less than the characteristic area such as, for example, areas that are less that 1/10, 1/100, 1/1,000, or 1/10,000 times the characteristic area.

In representative examples described herein, selection of RNA molecules for gold nano-crystal synthesis is based on in vitro RNA selection using the co-called Systematic Evolution of Ligands by Exponential Enrichment (SELEX) method. In a typical conventional example, a mixture of 10¹⁵ or more random RNA sequences is combined with gold flakes, so that the at least some RNA sequences preferentially bind to the gold surfaces. These bound RNA sequences are amplified to obtain a second RNA mixture that can be combined with gold flakes again. The bound RNA is typically amplified using a reverse transcription polymerase chain reaction (RT-PCR). In RT-PCR, an RNA strand is reverse transcribed into its DNA complement or complementary DNA, and is amplified using a polymerase chain reaction. Binding of the selected RNA can be evaluated to determine if additional selection and/or amplification steps are necessary. The SELEX method is described in Gold et al., U.S. Pat. No. 5,705,337 and Gold et al., U.S. Pat. No. 5,270,163 which are incorporated herein by reference. These methods typically exhibit inadequate surface selectivity and do not provide appropriate concentration control. Preferential selection based on surface binding is also referred to as In Vitro Selection on Surfaces (ISOS). The process of repetitive selection can be limited by the competitiveness of binding to the target (such as gold flakes). Superior results can be obtained using the methods and apparatus described below.

While a particular example is described in detail below, in other examples, other magnetic and metallic crystals such as Co, CoPt, and FePt can be formed using candidate molecules as selected as described herein. Conventional techniques for metal or semiconductor nano-particle fabrication are described in, for example, Gugliotti et al., J. Am. Chem. Soc. 127:17814-17818 (2005) and Whaley et al., Nature 405:665-668 (2000). Representative materials include GaAs, AlGaAs, CdS, GaN, ZnS, and SiO₂.

### Representative Reaction Vessels

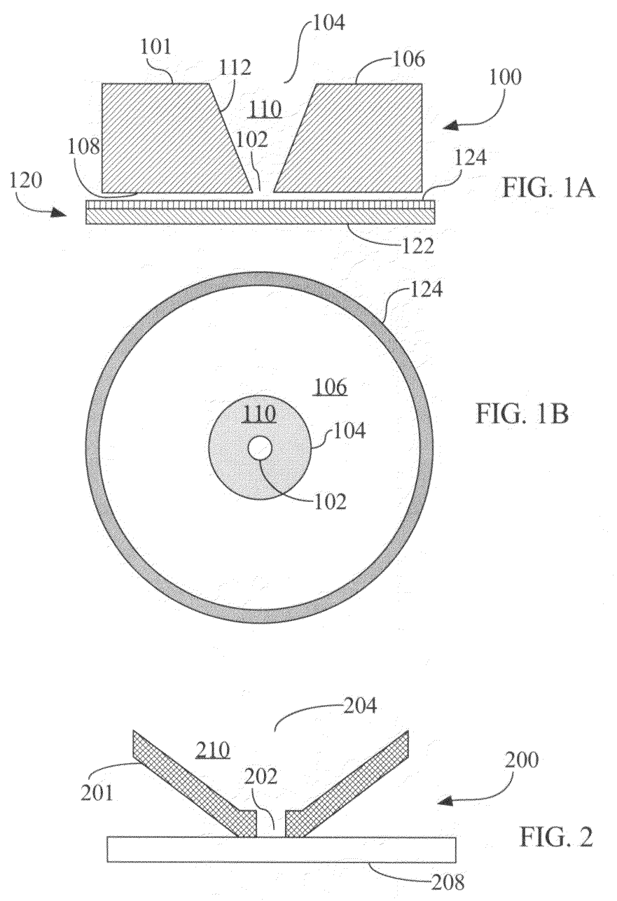

FIG. 1A is a sectional view of a representative reaction vessel 100, and FIG. 1B is a plan view. Typically such a reaction vessel is coupled to one more additional components to provide for convenient reagent introduction, observation, temperature control, or to provide additional mechanical support. Such combinations are generally referred to herein as “reactors.” Referring to FIGS. 1A-1B, the reaction vessel 100 is formed in a cylindrical block 101 having apertures 102, 104 on opposing major surfaces 106, 108, respectively. The major surfaces 106, 108 are conveniently substantially parallel but need not be. A volume 110 is defined in the block 101 by a tapered surface 112 and the apertures 102, 104. As shown in FIGS. 1A, 1B, the apertures 102, 104 are substantially circular and have respective radii R₁, R₂ that define aperture areas A₁, A₂.

Typically, the block 101 is formed of a chemically inert material such as polychlorotrifluorethyene (PCTFE) or other or fluoropolymers, or other materials including glasses, plastics, and metals or other solids. PCTFE is convenient because it is mechanically stable, readily machined or otherwise shaped, and is resistant to moisture and chemical attack. In addition, PCTFE has a substantial useful temperature range of between about −240 degrees C. and +200 degrees C. In addition, many potential materials used in ISOS exhibit little specific or non-specific binding to PCTFE, so that PTCFE does not interfere with the ISOS process.

Referring again to FIGS. 1A-1B, the reaction vessel 100 is situated adjacent to a target substrate 120. The target substrate 120 includes a support plate 122 and a target surface 124. In some examples, the target surface 124 is a gold surface configured as a substantially common crystalline gold surface. The target substrate 120 and the reaction vessel 100 are situated so that the reagent volume 110 terminates at the target surface 124.

The reaction vessel 100 and the target surface 124 can be selectively arranged to enhance, suppress, or otherwise control, regulate, or influence competitive binding to the target surface 124. Reagents that are introduced into the reagent volume can include components that preferentially bind to the target surface 124. Because only the target surface 124 is available for preferential binding by all constituents of the reagent, the area A₁ of the aperture 102 can be selected based on reagent volume, relative concentrations of various constituents of the reagent, or total numbers of constituent molecules or other structures so to promote preferential binding to the target surface 124. For example, if the selected area A₁ of the target surface 124 includes at most about N preferred binding sites, the candidate mixture can be configured so that the mixture includes substantially more than N candidates to promote competition for the available binding sites. Such an arrangement tends to reduce the quantity of untargeted constituent that becomes bound to the target surface 124.

A gold target surface is convenient for the selection of materials for the production of gold nanoparticles. Such preferentially selected materials can be used to cover or partially cover selected surfaces of gold particles so as to inhibit particle growth at the covered surfaces. However, the target surface can be arranged to provide additional selectivity based on, for example, an orientation of atoms, molecules, or other constituents of the target surface. For example, a gold surface can be selected that represents a single gold crystal surface. In this manner, the selected process can be both material specific, and crystal surface specific. In other examples, a random arrangement of a gold target surface can be used to reduce selection sensitivity to gold crystal structure. In some examples, a gold target surface is produced by sputtering, evaporation, atomic layer deposition, electroplating, or other process and optionally processed to so as to provide a preferred crystalline arrangement.

While the configuration of FIGS. 1A-1B is convenient, other reactor arrangements can be provided. Referring to FIG. 2, a reaction vessel 200 is defined by a tapered funnel-like section 201 that is terminated by apertures 202, 204. The section 201 can be formed of materials such as PTCFE or other materials, and an overall height of the section 201 and the apertures 202, 204 can be selected to provide a predetermined reagent volume 210. A target substrate 208 adjacent the section 201 is also shown in FIG. 2.

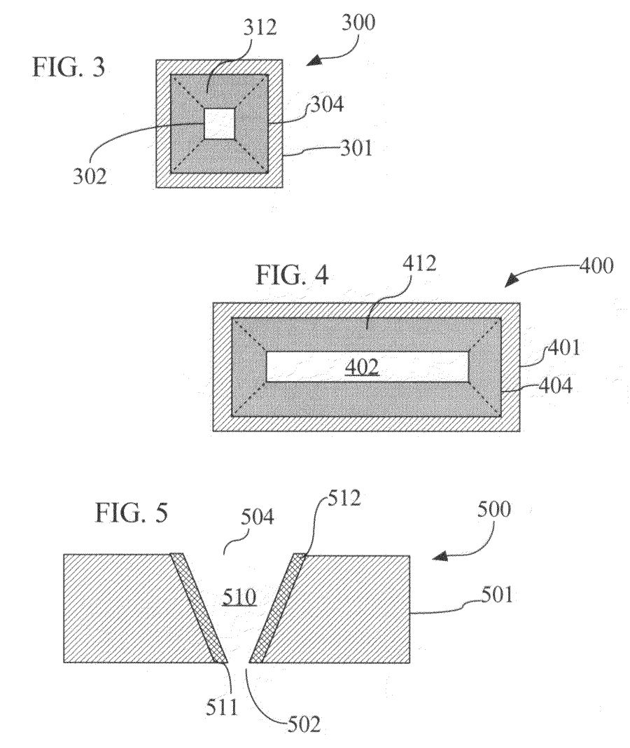

In other examples, apertures and reagent volumes are defined by square, rectangular, or other apertures. For example, FIG. 3 is a plan view of a reaction vessel 300 defined in square or rectangular block 301. A square tapered bore 312 terminates in square apertures 302, 304. FIG. 4 is a plan view of an example in which a reaction vessel 400 is defined in a rectangular block and includes a rectangular tapered region 412 that terminates at apertures 402, 404.

While unitary or “one-piece” construction is convenient, reaction vessels can also be made of two or more pieces. For example, FIG. 5 is a sectional view of a reaction vessel 500 that includes a support member 501 having a tapered bore that terminates at apertures 502, 504 to define a reagent volume 510. However, a surface 511 of the support member 501 that would otherwise contact reagents in the volume 510 is covered with a conforming insert 512. The insert 512 can be rigid, or semi-rigid, or can be supported primarily by the support 501. In addition, the insert 512 can be permanently secured to the support 501, or can be removable so that the insert 512 can be cleaned or otherwise processed or replaced independently of the support 501. In some examples, the insert 512 can be a conformable film that is applied to the surface 511. Removable or disposable inserts can be conveniently used to promote selective binding of candidates that are to be excluded.

Although not shown in the drawings, aperture shapes other than circular or square can be used such as, for example, oval, elliptical, rectangular, polygonal, oval, or other shapes as convenient. Aperture cross-sectional area can be varied based on the selection process. For circular apertures, typical areas are those associated with aperture diameters of between about 0.5 μm and about 5 mm that are conveniently fabricated for laboratory apparatus. Larger or smaller diameters can also be used.

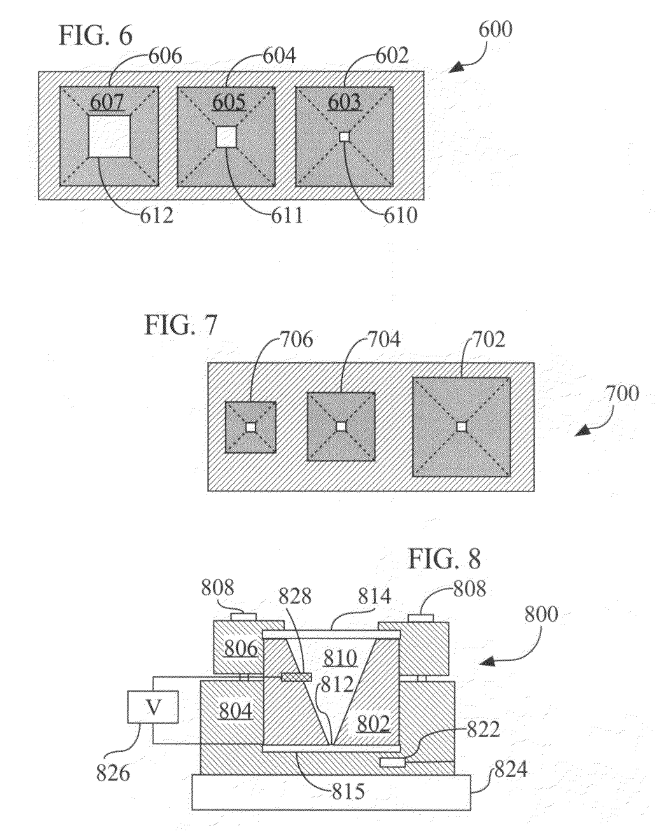

In some examples, one or more reactor volumes can be defined on a single reaction vessel substrate. Referring to FIG. 6, a plurality of reactor volumes are defined by square tapers 603, 605, 607, reagent introduction apertures 602, 604, 606 and target exposure apertures 610, 611, 612, respectively. As shown in FIG. 6, the reagent entrance apertures are all of the same size, and the target exposure apertures vary. Therefore, the capacity of the reagent volume also varies. In an additional example shown in the plan view of FIG. 7, a reaction vessel 700 includes three sample volumes 702, 704, 706 having different volumes and terminate in a target exposure aperture of a common size.

### ISOS Reactors

In many examples, temperature selection and reagent confinement can be provided for additional control. Referring to FIG. 8, a reactor 800 includes an insert 802 that defines a candidate volume 810 that is terminated by an exposure aperture 812 and a window 814. A target substrate 815 is situated at the exposure aperture 812, and the window 814, insert 802, and target substrate 815 are secured to each other by first and second retention blocks 804, 806. The candidate volume 810 can be provided as a tapered bore in a cylindrical section of a suitable material such as PTCFE. Screws 808 or other fasteners are provided to urge the retention blocks 804, 806 toward each other. The retention blocks 804, 806 are typically formed of a conductive material such as a metal, and a thermocouple 822 or other temperature sensor is provided to permit temperature control of reagents in the candidate volume 810 and/or a target surface. A heating element 824 is coupled to the retention plate 804. Control circuitry coupled to the heating element 824 and the thermocouple 822 are not shown in FIG. 8. In some examples, electrochemical stringency is used by applying a bias between the substrate and a counter-electrode inserted into the reaction vessel. As shown in FIG. 8, a voltage source 826 is coupled to the target substrate 815 and to the reaction volume via an electrode 828 through the insert 802.

For convenient selection of a target aperture size, a plurality of inserts can be provided, each having a different target aperture. If such inserts have different outside diameters, a spacing sleeve or other spacer can be positioned between the insert 802 and one or both of the support structures 804, 806. A circular cross-section is simple to fabricate, but reaction vessels of square, rectangular, elliptical, or other shapes can be used. Alternatively, a plurality of inserts of different volumes can be provided, or inserts having both differing volumes and target apertures.

### Representative ISOS Methods

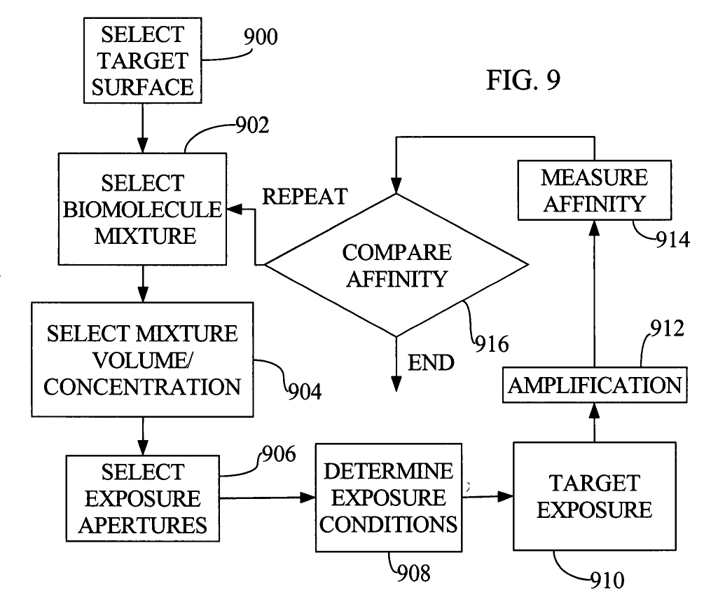

A representative method is illustrated in FIG. 9. In a step 900, a target material (such as gold) and/or target surface (a particular crystal surface of the selected material) is selected. In a convenient example, a (111) gold surface is selected. In a step 902, a mixture of RNA sequences or other biomolecules is selected and in a step 904, a total mixture volume is selected. A total number of random sequences can be chosen based on a sequence concentration, and the mixture can be an initial mixture or a previously processed mixture. Based on the selected volume and/or mixture concentration, one or more exposure aperture areas are selected in a step 906. Additional exposure conditions are determined in a step 908 such as, for example, reaction temperature, exposure duration, or reaction mixture pH. In some examples, reaction vessel surface material can be selected to, for example, reduce RNA sequence binding to the surface. Alternatively, the surface material can be selected to enhance binding of RNA sequences that are preferably removed from the mixture. In a step 910, the RNA sequence mixture is introduced into a reaction chamber, and exposed to the target surface under the previously determined selection conditions.

Upon exposure completion, in a step 912 the RNA species bound to the target surface are amplified by RT-PCR to provide a second RNA mixture. The affinity of this RNA mixture with respect to the target surface is measured or estimated in a step 914. Based on this affinity, in a step 916 a determination can be made as to whether to repeat the selection process. Typically, reaction conditions, target surfaces, and other parameters similar to or the same as used in a first selection process are used in subsequent selection steps, but different conditions can be selected.

In the step 914, the affinity of the amplified, selected RNA strands is determined. In some cases, the selection process produces an affinity that approaches an asymptotic or other limit so that additional steps produce increasingly smaller improvements. In these examples, the selection process is typically terminated although some slight improvements can be realized with additional steps. In other examples, large improvements in selectivity can be obtained with additional steps, but the achieved selectivity is already satisfactory so that the selection process can be terminated.

In some examples, a number of target exposure steps is determined by using the amplified, selected candidates for the intended application. For example, a candidate mixtures can be used to promote a predetermined gold crystal shape. In one example, candidate mixtures produced in four rounds of processing can show little difference in a particular application, but fifth and sixth rounds can produce significant differences. Thus, performing a certain number of selection steps can be useful, even if the initial steps do not appear to be offering an improvement, and evaluation based on actual use in an intended application increases the reliability of candidate assessment.

In a particular example, a target surface is a gold surface. A target surface area is selected to provide about 120 pmoles of binding sites, and is exposed to a mixture of about 600 pmoles of candidate RNA. The selected candidates are extracted and amplified, and an additional exposure/extraction/amplification step is performed with substantially the same numbers of binding sites and candidates. In these two steps, a ratio of a number of candidates to number to a number of binding sites on the target surface is about 5. In two subsequent steps, about 40 pmoles of binding sites are exposed to about 120 pmoles of candidate RNA. In two final steps, about 4 pmoles of binding sites are exposed to about 40 pmoles of candidate RNA. While in this examples, binding site/candidate ratios range from about 1:3 to about 1:10, different ratios can be used. In addition, processing conditions such as temperature can be varied for one or more exposure steps. Typically the selected aptamers are evaluated at each or some processing steps in the intended application.

While ratios of candidate biomolecules to binding sites of 2:1 or greater are used in the above examples, ratios of between about 1:1000 and about 1000:1 are often convenient. Exposure surface areas can also vary widely depending on available reactors exposure apertures, and areas from at least as small as about 1 μm² to about 1 m² are convenient

In some applications, in an initial step, a target surface is not used, and candidates that bind to reaction chamber materials can be eliminated, or concentrations thereof reduced. For some applications (such as formation of gold nano-particles), the selected candidates generally are to be used in a reducing solution. For this reason, the selection process can be carried out in the presence of any necessary reducing agents or at a preferred pH. For gold nano-particles, ascorbic acid can be used to enhance gold particle growth, and candidates can be exposed to a target surface in solution that includes ascorbic acid. Thus, the competition process is carried out under conditions to those of the intended application.

For candidate selection for gold nano-particle fabrication, the target surface can be a (111), (100), or (101) gold surface. Applying the selected RNA tends to passivate the corresponding gold seed crystal surface. (111) passivation tends to produce nanoprisms, while (100) passivation tends to produce nanorods.

Target materials can be provided on planar substrates such as, for example, thin metallic or other layers formed on a glass or quartz substrate. Portions of crystalline substrates can be used as a target surface or a support surface that promotes a selected crystalline or other orientation of a target material grown, deposited, or situated on the support surface.

As noted above, the ISOS method has been applied to a variety of material systems, and the disclosed methods and apparatus can be used with conventional materials systems or applied to candidate selection for additional applications. Typical target surfaces suitable for use with the methods and apparatus described herein include organic, inorganic, metallic, semiconductor, or biopolymer target surfaces. For crystalline materials, a particular crystalline surface can be used as a target surface. For example (111), (110), or (100) surfaces (or combinations) can be used. RNA can be selected having a high affinity for one or more crystal surfaces, as preferred in a particular application. While peptides can be similarly selected for use in, for example, nano-material fabrication, RNA mixtures typically provide greater diversity of candidates and higher specificity.

While representative examples of the disclosed technology are described in detail herein, it will be appreciated that the disclosed examples not to be taken as limiting the scope of the technology. We claim all that is encompassed by the appended claims and the equivalents thereto.

1. A reactor for selective enhancement reactions, comprising:
a reactant chamber that includes a reagent introduction aperture, and a target exposure aperture, wherein the reagent introduction aperture has an area at least ten times greater than an area of the target exposure area.

2. The reactor of claim 1, wherein the reactant chamber is defined by the reagent introduction aperture, the target exposure aperture, and reactant chamber walls, wherein the reactant chamber walls are formed of a material that tends to exhibit low affinity for candidate molecules situated in the reaction chamber.

3. The reaction vessel of claim 3, wherein the reactant chamber walls are formed of a fluoropolymer.

4. The reactor of claim 3, wherein the target aperture has an area of less than about 100 mm².

5. The reactor of claim 3, wherein the target aperture has an area of less than about 10 mm².

6. The reactor of claim 3, wherein the target aperture has an area of less than about 1 mm².

7. The reactor of claim 3, further comprising;
a target support configured to support a target surface for coupling to the target aperture; and
a temperature sensor thermally coupled to the target surface and the target support, and configured to be responsive to a temperature of the target surface.

8. The reactor of claim 6, further comprising a heating element coupled to the target surface and responsive to the temperature sensor so as to establish a temperature of the target surface.

9. The reactor of claim 1, further comprising at least two target exposure apertures of substantially different areas.

10. A method, comprising:
selecting a volume of a candidate molecule mixture;
exposing the candidate molecule mixture to a target surface through an aperture, wherein the aperture area is less than ⅕of a characteristic surface area of the candidate molecule mixture.

11. The method of claim 8, wherein the aperture area is less than 1/50of the characteristic surface area.

12. The method of claim 8, further comprising, selecting a concentration of candidate molecules in the candidate molecule mixture based on the aperture area.

13. The method of claim 8, further comprising selecting a number of candidate molecules in the candidate molecule mixture based on the aperture area.

14. The method of claim 8, further comprising selecting an aperture area based on a concentration of candidate molecules in the candidate molecule mixture.

15. The method of claim 8, further comprising selecting an aperture area based on a number of candidate molecules in the candidate molecule mixture.

16. The method of claim 8, further comprising configuring the target surface to be a single crystal surface.

17. The method of claim 15, wherein the target surface is a (111) gold surface.

18. The method of claim 8, further comprising:
extracting candidate molecules attached to the target surface;
amplifying the extracted candidate molecules;
preparing a supplemental candidate molecule mixture based on the amplified, extracted candidate molecules; and
exposing the supplemental candidate molecule mixture to the target surface.

19. A reactor for selective enhancement of candidate molecules, comprising:
a candidate mixture chamber coupled to a target exposure aperture and an mixture introduction aperture, the chamber defined by a tapered bore in a section of a cylinder, wherein a target aperture radius is less than ¼of a radius of the cylindrical section;
a cover plate;
a support structure that includes a top portion, a bottom portion, and at least one connector, wherein the bottom portion is configured to receive the cylindrical section and the top portion is configured to contact the cover plate so as to substantially seal the chamber from the mixture introduction aperture, and the connector is configured to secure the top portion and the bottom portion.

20. The reactor of claim 17, wherein the cylindrical section is a fluoropolymer.

21. The reactor of claim 18, wherein the fluoropolymer is polychlorotrifluorethyene.

22. The reactor of claim 18, wherein the window is transparent.

23. The reactor of claim 18, wherein the target aperture is circular and has a radius between about 500 μm and 2 mm.

24. A selective enhancement method, comprising:
selecting a ratio of candidate molecules to target binding sites, wherein the ratio at least 2:1;
selecting a portion of a target surface based on the ratio; and
exposing a candidate mixture to the portion of the target surface, wherein the candidate mixture includes a candidate molecule concentration based on the ratio and the selected portion of the target surface.

25. The method of claim 22, wherein the ratio is at least about 4:1.

26. The method of claim 22, wherein the ratio .is at least about 10:1.

27. The method of claim 22, further comprising exposing the candidate mixture to the target surface based on exposure conditions associated with passivation of a surface by selected candidates.

28. The method of claim 25, wherein the exposure condition is pH.
